(19) United States
(12) Patent Application Publication
(10) Pub. No.: US 2008/0057090 A1
(43) Pub. Date: Mar. 6, 2008

(54) Wrinkle masking film composition for skin

(76) Inventors: Edward Enns McEntire, Kingsport, TN (US); Rebecca Reid Stockl, Kingsport, TN (US); Ramesh Chand Munjal, Kingsport, TN (US); Vicky Lynn Christian, Mount Carmel, TN (US)

Correspondence Address: Brett L. Nelson;Eastman Chemical Company, P.O. Box 511, Kingsport, TN, 37662-5075, US

(21) Appl. No.: 11/515,150

(22) Filed: Sep. 1, 2006

(51) Int. Cl. A61K 8/02

(52) U.S. Cl. 424/401

(57) ABSTRACT

A film product composition that has a refractive index that matches the refractive index of the skin. The composition is made from polymers, polymers plus additives, including plasticizers, which have an average effective or actual refractive index of from 1.4 to 1.6. The refractive index of the composition matches the skin's refractive index, and therefore is helpful at masking skin fissures and imperfections such as wrinkles, cracks, abrasions and the like.

### FIELD OF THE INVENTION

The present invention relates to compositions for use on skin and methods of making and using the compositions.

### BACKGROUND

Conventional makeup is often used to hide wrinkles in skin, but with limited success. When applied, makeup such as concealer provides a different, often matted appearance, and therefore unnatural skin texture, noticeable to observers. The makeup color is also somewhat different than the underlying skin, giving then an unnatural color. This combination of texture and color differences may show some improvement to the underlying skin, but are not perceived by the observer as natural. In addition, makeup does not form a substantial polymer film and results only in large amounts of pigments applied to the skin, held together loosely by polymer and other ingredients. These makeup formulas contain generally less than 5% polymer by weight, and the polymer contributes little other than to bind the pigments in place. Water and oil resistance is typically not excellent. Makeup is often applied from organic solvents which can be harsh for the skin. Application from water is preferable.

While many particulate materials have been used with refractive indices of from about 1.2 to over 4 for incorporation into products for the skin, and one reference claiming a polymer film composition having a desirable refractive index of 1.1 to 1.4, preferably 1.2 to 1.3 (US Patent Application 20030095941 A1 Cont of U.S. Pat. No. 6,491,929) to form an antireflective layer forming material for the skin, none has claimed the desirability of matching a coating composition refractive index to that of the skin.

### SUMMARY OF THE INVENTION

An embodiment of the present invention concerns a cosmetic composition, that includes at least 5% by weight of a film forming polymer and a cosmetically acceptable carrier. After the composition is applied to and dried on skin, the composition has a refractive index (hereinafter referred to as RI) of between about 1.4 and about 1.7.

Another embodiment according to the present invention concerns a method of making a cosmetic composition. The method includes combining a film forming polymer and a cosmetically acceptable carrier. The film forming polymer is present in an amount of at least 5% by weight, and after the composition is applied to and dried on skin, the composition has an RI of between about 1.4 and about 1.7.

Yet another embodiment concerns a method of using a cosmetic composition which includes applying the composition according to the present invention to skin.

### DETAILED DESCRIPTION OF THE INVENTION

To gain invisibility on the skin and yet preserve the natural skin pigmentation, the present invention uses a polymer matched to the refractive index of the skin, so that the skin-polymer film interface is rendered invisible to the eye, and the eye sees only the upper film surface (air-polymer film interface). Since the polymer film is largely transparent, the original skin coloration underneath the film is preserved. In order to minimize shine, it is desirable that a particulate material be incorporated, which may be colored similarly to match the skin or even clear, even having the same RI as the film formed on the skin, but having a particle diameter of from about 1% to about 100% of the dried film thickness so that the film surface is not smooth. The polymer film of this invention may take the place of makeup, or makeup may be used over the polymer film.

The present inventors have found that a film product that has a refractive index that matches the refractive index of the skin is indeed helpful at masking skin fissures and imperfections such as wrinkles, cracks, abrasions and the like. We have also found that particulate matter of refractive index different than the film also helps soften the look of the film on the skin and further hide the skin imperfection or wrinkle over which it is applied. Contrary to US Patent Application 20030095941 A1, continuation of U.S. Pat. No. 6,491,929, rather than the polymer to form the film being formed by polymerizing monomers on the skin, the film of the present invention is formed by applying a previously formed polymer from a liquid (to include paste or gel) solution or dispersion. It is advantageous not to polymerize monomers on the skin. For example, polymerizing monomers emit heat which may be uncomfortable, monomers may be absorbed into the skin and may cause trauma to the skin, or may emit organic materials not desirable for human exposure or to the environment. In addition, polymerization on the skin to achieve the exact film thickness needed is problematic. To form the desired film thickness may be difficult using the methods suggested by 20030009594 A1.

The refractive index of the skin is thought to be between about 1.4 and 1.7, and close to 1.55, although some variation will occur within skin types, in the amount of hydration of the skin, and variation of skin chemistry (natural skin chemicals such as squalene, salts, natural moisturizing factor [NMF], and the like may vary in concentration within the skin depending on perspiration, skin cleanliness, and other factors unknown). These variations may be responsible for the reported values of skin refractive index of from 1.4 to 1.7. Thus, films made from polymers, polymers plus additives, including plasticizers, which have an average effective or actual refractive index of from 1.4 to 1.6, match the skin's refractive index, and therefore hide the skin surface. Suitable films are those films formed on the skin comprising polymer plus additives which have a refractive index of 1.5 to 1.6, from 1.52 to 1.58, or even from 1.53 to 1.57.

The polymer may be delivered from a cosmetically acceptable carrier. Examples of cosmetically acceptable carriers include a solvent such as water, ethanol, isododecane, cyclomethicone and the like, and mixtures thereof. The polymer may be soluble or dispersible in the solvent. A surfactant may be used to aid in the dispersion of the polymer and other ingredients in the solvent. The delivery may also be rendered from an emulsion, oil-in-water or water-in-oil, or a multiphase emulsion, where one phase contains water.

Films should be of a thickness on the skin so that they provide hiding of wrinkles. Guidelines for thickness are that the film should fill or partially fill gaps or crevices within the wrinkle. Thus the depth of the wrinkle should be filled to at least about one fourth of its depth as measured from the deepest part of the wrinkle to its surface by the dried film. The thickness of the dried film on the skin may be from about 0.2 to about 5 mil (thousandths of an inch, or 5 micrometers to about 125 micrometers), or even from about 0.5 to 4 mils (about 12 to about 100 micrometers). These film thicknesses help the film be imperceptible by feel on the skin. The film should also be elastic for most skin applications. For skin surfaces that flex, such as knees, elbows, around the eyes, on the hands (especially the knuckles) and feet, the elongation of the film should be at least 50%, 100%, or even 150% or more as measured by ASTM Method D882 under the conditions stated: for a dry film thickness of from 0.6 to 0.7 mils and when evaluated following an ambient temperature cure at 50% relative humidity for 24 hours.

### Film Gloss and Color

The film may be clear and colorless or lightly pigmented or dyed to aid in skin matching. Particulate material may also be used to aid in rendering the film imperceptible by vision. The particulate material may be added into the liquid polymer composition which when dried to a film is caused to have low gloss and some opacity or haziness. That is, the film, if cast onto a bare aluminum Q panel—Type A (available from Q-Panel Lab Corporation, Cleveland, Ohio 44145) using a 3 mil gap 8-Path Wet Film Applicator (available from P. D. Gardner Company, Pompano Beach, Fla. 33060) have a 60° gloss of from about zero to about 20 as measured with a Micro-TRI-gloss meter (available from P. D. Gardner Company, Pompano Beach, Fla. 33060), a gloss of less than 15, or even a gloss of 10 or less. Ideally the gloss of the film should match the gloss of the skin.

Particulate materials with a RI similar to that of the dried film of a particle size of from about 0.1 to about 250 micrometers in diameter are suitable for reducing gloss and adding haze to the film. Particles having a mean particle diameter from about 0.5 to about 200 micrometers, or even from about 1 to about 150 micrometers are also suitable. Preferred for skin feel upon application are particles with diameter about 30 micrometers or less.

Particulate materials with a RI substantially different to that of the dried film of a particle size of from about 0.1 to about 30 micrometers in diameter are suitable for reducing gloss and adding haze to the film. Particles having a mean particle diameter from about 0.5 to about 20 micrometers, or even from about 1 to about 15 micrometers are also suitable. It is preferable that the particle size distribution range of these non-index matching particles distribution not extend too high in diameter since said particles will be too large and cause whiteness in the film or too great of opacity, and particles too small have a much smaller influence on gloss reduction. Experimentation with such particulate materials with an exact formula by one skilled in the art is the best method of particulate material selection to achieve a desired film appearance.

Particulate materials may be inorganic or organic. Moreover, the particle composition may be any of the common extender pigments such as silica, calcium carbonate, alumina, clay, synthetic or natural silica-alumina, talc, coated silica, coated alumina, coated carbonates and the like. The particles may also be hollow particles. The particles may be organic particles coated with inorganic material or inorganic particles coated with organic material. The particles may have a refractive index the same or different than the dried film. If the refractive index for the dried film and the particles are similar the coating will be clear, but the film gloss will still be reduced.

The particulate material can be spherical in form, but plate-like or irregular shapes are also suitable. Ideally the color of the particulate material is colorless and transparent, but may be white until it is wetted by the film composition. Dye or finely ground pigment may be dispersed into the liquid composition to aid in matching skin color. Disruption of the film surface is desirable as a way to prevent film gloss so that the film is imperceptible on the skin. If the refractive index of the particulate matches the refractive index of the dry film, then only surface roughness will contribute to gloss reduction. If the refractive index of the particulate is very different than that of the dry film, then internal reflections and other optical phenomena will also contribute to the overall appearance of the film. Other suitable methods of disrupting the film surface smoothness are acceptable for film gloss reduction.

The refractive index of the polymer film will be influenced by other added ingredients. Therefore, in preparing the composition, one must take into account their refractive index influence on the dry film. Also the time of residence of these added ingredients in the film must be considered—for example, an active ingredient or solvent or plasticizer may evaporate or be absorbed into the skin, thus being removed from the polymer film. This may cause a shift in the refractive index and consequently in the amount of wrinkle apparent, fading from deep to shallow wrinkles, or shallow to deep wrinkles, for example, with time.

The liquid composition may be applied by many different methods; brushing, spraying, wiping, smearing, spreading are acceptable methods of application. The composition may be in the form of a liquid pourable at 20 degrees Celsius, or may be in the form of a gelled liquid. The viscosity may be from about 1 to about 2000 centipoises as measured by a Brookfield viscometer at a shear rate of from 0.1 to 1000 sec-1. If the composition that is applied to dry as a polymer film is in the form of a spreadable lotion, cream, or gel, the viscosity should be from about 500 to about 10,000 centipoises measured as above, and the cream or gel should have an appropriate yield stress to provide a low spreading viscosity.

Imperceptibility of the film for the purposes of this invention is intended to include that the film is imperceptible visually, feel by the wearer of the film, and odor. To the wearer of the liquid applied film, the only sensations should be cooling during drying as the solvent evaporates to form a film and a faintly perceptible and not unpleasant tightening at or near the area to which the film is applied. To an observer, the film will be nearly or completely unnoticed, not perceptible visually. However, there may be applications in which a glossy film is desired, so only a shine would be obvious.

### Polymer Composition

Polymers that can be used to form the film are those with refractive indices of close to 1.55. Sulfopolyesters and polyesteramides are particularly suitable polymers and are water dispersible. These polymers are described in the following U.S. Pat. Nos. 3,734,874; 3,779,993; 3,828,010, 4,233,196, 5,006,598, 5,543,488, 5,552,511, 5,552,495, 5,571,876, 5,605,764, 5,709,940, 6,007,749 and 6,162,890, the disclosures of which are incorporated herein by reference. Other references describing closely related sulfopolyesters or sulfopolyamides also suitable are R. Breitenback, et. al., U.S. Pat. No. 6,036,962 and R. A. Hayes, et. al., U.S. Pat. No. 6,746,779.

Whereas others have used sulfopolyesters in emulsion formulas for anti-wrinkle purposes, the said polymer was used as a polymer for retensioning the skin and the formula required tensioning polymer particles and an amphiphilic ionic polymer (Cassin, US Patent Application 2004-0136937). Only small amounts of sulfopolyester polymer, however, were used in the compositions, 2% at most, in an example. Many other types of polymers were also used as retensioning agents. Furthermore, surfactants were limited to 1% or less. No mention of polymer or composition refractive index was made, nor were plasticizers used or recommended with high Tg polymers (Tg 55° C. in the example). In the example the sulfopolyester was used with a second polymer, which would cause the refractive index of the film to depart from the ideal refractive index of the sulfopolyester alone.

U. S. Patent Application 2004-0136937 by Cassin reveals the use of sulfopolyesters, poly(2-acrylamido-2methylpropanesulphonic acid), acrylic or acrylic copolymers or urethane polymers on the skin in combination with an amphiphilic ionic polymer and in the substantial absence of surfactants (<1%) for anti-wrinkle purposes. The said polymers are described as a retensioning agent. Only a small percentage (2% shown in Example 4) of the sulfopolyester is used in a formula, and a maximum of 7% of any single polymer (polyurethane, etc) is used in any emulsion example, even though from 1% to 50% is taught in the specification. We have surprisingly found that these polymers function as delivery agents for active ingredients without amphiphilic polymers and optionally in the presence of plasticizers and surfactants. Cassin reported no plasticizer use with any polymers. The thickness of any polymer film formed on the skin as taught by Cassin is at most 2.7 micrometer (0.11 mil) (for Example 2 film) since the solids of the film are approximately 13.3% (since “2 mg per square centimeter of the test composition are applied to the stratum corneum,” which would result in the 0.11 mil coating thickness), much thinner than the films of this invention. No mention of polymer or composition refractive index was made, nor were plasticizers used or recommended with high Tg polymers (Tg 55° C. in the example). In the example the sulfopolyester was used with a second polymer, which would cause the refractive index of the film to depart from the ideal refractive index of the sulfopolyester alone.

Acrylic polymers are suitable for use in this invention. Also acrylic hybrid polymers wherein acrylic polymers are polymerized in the presence of sulfopolyesters are also suitable and are described in U.S. Pat. No. 6,001,922 incorporated herein by reference. Naturally, the acrylic monomers used to prepare the polymers must be chosen to provide the desired refractive index and other desired film properties. Similarly, conventional acrylic polymers are acceptable, whether formed in emulsion or solution polymerization.

Polyurethanes containing water dispersing groups are also suitable. Water borne polyurethanes, depending on their composition, may have refractive indices very close to 1.55, and thus need little in the way of additives to shift the film refractive index close to 1.55.

Polymers useful in this invention should be removable from the skin once applied by water washing or peeling. Additives such as surfactants, water soluble salts, water soluble organic materials such as glycerin, propylene glycol, and other humectants may be added to both increase water removability, but also to improve flexibility or adhesion through their plasticizing influence. Other polymers may be added to adjust skin adhesion, water solubility, or formulation viscosity, such as silicone polymers, polyethylene glycol, polyacrylamide and hydrophobe modified hydrophilic polymers.

It is often difficult to achieve the desired refractive index of the film with many conventional polymer types. This is often due to the inherent refractive index of the polymer being far away from the typically most desired value of 1.55. For example, polymers prepared by free radical polymerization of commercially readily available acrylic monomers cannot achieve the desired target due to the low refractive index of the resultant polymers (for example poly(methylmethacrylate) has a refractive index of 1.489). Only in special cases can the desired refractive index be achieved, for example, by incorporating monomers with high refractive index content such as those with high aromatic content such as benzyl acrylate, phenoxyethyl acrylate, benzyl methacrylate and the like. A limitation of this approach is that to achieve the target 1.55 refractive index for the polymer, a relatively large amount of the aromatic monomer is required, often greater than 50%, and when these amounts are present, the Tg and expense of the polymer is generally increased, resulting in a higher cost than desired polymer of poorer than desired flexibility and elongation.

Another exception to the use of acrylic type monomers is that polymers containing about 50% styrene may also contain acrylic monomers and still achieve the desired refractive index target. Again, due to the large amount of the high Tg monomer, styrene, co-monomers must be chosen wisely to achieve both the refractive index desired and a polymer Tg desirably below 80 degrees C., below 70 C or even below 60 C. Polymers with Tg's as high as the numbers noted just above, typically require the use of a higher than desired amount of plasticizer to gain the needed toughness and flexibility for use on the skin. In the case where two types of aromatic monomers are employed in a polymer, such as styrene and phenoxyethyl acrylate, for example, then each individual monomer may be significantly less than 50%. The approximate level of aromatic monomer to incorporate to achieve the desired refractive index may be readily calculated by one skilled in the art.

Another example of polymers commonly used on the skin is copolymers of vinyl pyrrolidinone and aliphatic olefins. These would not be as suitable as the sole film component due to the inherent low refractive index of the polymer. Poly(1-vinyl-2-pyrrolidinone) itself gives a homopolymer with a refractive index of 1.53, whereas when copolymerized with olefins the refractive index is even lower.

Polymers such as hydroxyethyl cellulose are generally not suitable to match the RI of the skin. This polymer's RI is 1.51, yet it readily absorbs moisture from skin and the air, and water has a refractive index of 1.33, thus significantly decreasing the overall refractive index of the formed film.

Silicone polymers are used frequently on the skin, but most are of such low refractive index due to the nature of the typical silicone linkage, that they are of little use in achieving a refractive index near 1.55. These are typically polymers containing mainly dimethyl silicone units. However, polymers containing aromatic groups such as phenyl attached to silicon, such as phenyl methyl silicone polymers and diphenyl silicone polymers, may achieve refractive indices near enough to 1.55 to be suitable.

### Polymer and Film Tg

The Tg of polymers used in the skin film formers are desirably less than about 80° C., less than 70° C., or even less than about 60° C. The higher Tg polymers require plasticizer or solvent in such large amounts to maintain flexibility and elongation that film or application properties may suffer. Higher amounts of plasticizer are generally considered less suitable for skin application. Depending on the plasticizer, other concerns of stability may be an issue. For example, ester hydrolysis of ester linkage containing plasticizers can be a concern during preparation or storage, and often require close regulation of pH of the film forming dispersion. The most desirable plasticizers have a refractive index that brings the polymer film (containing plasticizer and additives) closer to the desired refractive index. Thus while simple esters are often effective plasticizers, those greater or equal to 1.5 are useful, such as diethyl phthalate, phenoxyethanol, phenoxypropanol, methoxyphenol, resorcinol, hydroquinone, and the like.

The compositions of this invention may be applied from a largely aqueous dispersion of polymer. The aqueous composition contains from about 5 to about 50 weight percent polymer, and may contain other ingredients such as plasticizer, surfactant, particulates (as referred to above), solvents such as ethanol, isododecane, and the like, silicones, and active ingredients for the purpose of treating the underlying skin with nutrients, moisturizing agents, vitamins, and the like. For example, the liquid composition should contain from about 5 to about 50% polymer by weight. It would also be suitable for the composition to contain from about 10% to about 40% polymer by weight. It would still also be suitable for the composition to contain from about 15 to about 35% polymer by weight.

To prevent the composition from being tacky upon application, or worse after drying, a limited amount of humectant may be present. We have found that based on the polymer present in the formulation, less than 20% of a humectant should be present (stated another way, for every 5 parts of polymer present, less than one part of humectant should be present). Less than 15% of a humectant is acceptable, and even less than 10% of a humectant is acceptable, based on polymer present in the formula. Thus for a formulation containing 25% polymer, less than 5% humectant should be present in the formula, or less than 3.75% of humectant is present, or even less than 2.5% humectant is present, all percentages in this sentence being based on the total formula composition.

Humectants are generally diols or polyols such as glycerol, sorbitol, propylene glycol, dipropylene glycol, diglycerol, polyglycerol, and the like. Another limitation of such humectants is that if too much is used, the RI of the film will be lowered by the humectant, so that the RI match to the skin is perturbed. Also, if too much plasticizer is used, a similar RI lowering may be observed, depending on the RI of the plasticizer. Generally the lower the polymer Tg, the less of an additive is used which may lower the RI lower than acceptable limits so that the wrinkle masking property is made less effective.

The film should be quick drying, drying easily to a tack free state within 15 minutes or less resulting in the desired film thicknesses. It would be desirable that the film is tack-free within 10 minutes, or even that the film is tack-free within 5 minutes. Its purpose of hiding wrinkles is fulfilled upon drying through refractive index matching the underlying skin. It would also be desirable that the refractive index (RI) of the dry film matches the skin refractive index to within 0.2 refractive index unit (RIU), or that the match is within 0.15 RIU, or even that the RI of the skin and polymer film matches to within 0.1 RIU or less.

### Composition Preparation—Emulsion Preparation

To prepare the liquid composition, optionally one or more active ingredients, such as petrolatum or such as glycolic acid, may be mixed with one or more emulsifiers and one or more water-based polymers. By the word “active ingredient” the inventors intend an ingredient to benefit the skin. Plasticizers, coalescing agents, solvents, oils, emollients, humectants, pigments, fillers, fragrance and other ingredients may be added to effect change in the properties of the wet mixture or of the dried film for either aesthetic and/or functional purposes. The mixture may spontaneously emulsify with water or may be homogenized using a high shear device. Devices such as high speed dispersers, rotor-stator mixers, impingement mixers and the like may be used, but often low shear mixing is adequate if the active ingredients or additives are liquid or properly liquefied. Depending on the system used, the mixture may be heated to soften, melt, dissolve or otherwise liquefy solid or waxy ingredients. The mixture is then homogenized by adequate mixing, usually shearing or stirring until it is cool if heated or subjected to high shear conditions causing heating.

Another procedure is to soften, melt, dissolve or otherwise liquefy solid or waxy ingredients by mixing with an oil or suitable liquid prior to adding that mixture with stirring to the water-based polymer and emulsifier. Heat may be used as needed. The mixture is then homogenized by adequate mixing usually until it is cool if the mixture has been heated.

Yet another procedure is to soften, melt, dissolve or otherwise liquefy solid or waxy ingredients by mixing the solid or waxy ingredients with one or more emulsifiers or surfactants prior to mixing with stirring into the water-based polymer. The surfactants may be anionic, cationic, amphoteric, or neutral. Liquids such as oils or suitable solvents maybe added to the mixture also. Heat may be used as needed. The mixture is then homogenized by adequate mixing usually until it is cool if the mixture has been heated.

The resulting homogenized mixture can then be applied directly to the skin, whereupon volatile materials evaporate to form a film. The resulting film can be removed by washing with water or by peeling, depending on the film thickness, solubility in and response to water, integrity, and the formulation ingredients used.

Yet another procedure is to mix the ingredients, including one or more dry polymers, before diluting the mixture with water and/or other liquids. The ingredients may be mixed uniformly using adequate agitation by heating and/or by adding liquids or suitable solvents and/or other polymers to liquefy all ingredients and/or by mechanically mixing with devices such as two-roll mills, extruders and sigma mixers, and other high sheer devices. The mixture may then be mixed with water and/or other liquids and/or other additives using adequate agitation and/or heat. This polymer ingredient blend may then be applied to the skin.

Yet another procedure is to mix one or more active ingredients with one or more monomers, such as styrene and 2-ethylhexylacrylate, and one or more emulsifiers and optionally one or more solvents, liquids, active ingredients to be delivered to the skin, or polymers, then homogenize the monomer mixture, then add an initiator so that the monomers are polymerized to form a polymer latex, typically called a mini-emulsion polymerization. The resulting miniemulsion contains ingredients such as active ingredients for treating the skin, which are incorporated into the formulation during the polymerization.

Regardless of the procedure used, the goal is to generate a uniform, stable emulsion or solution which, when applied to the skin, forms a film that hides wrinkles, and optionally delivers one or more active ingredients to the skin, yet acts as a barrier to help prevent active ingredients from transferring to adjoining fabric items, and is non-tacky as it resides on the skin.

Whereas much of the discussion has described aqueous organic emulsion produced films, similar films can be applied to the skin from solvent. To do this, one must dissolve or suspend the polymer and other ingredients in the organic liquid, then apply the formulation to the skin. No water need be present. Suitable organic solvents are those safe for skin application, many of which are known in the art, and have been described as ethanol, propanol, propylene glycol, glycerol, isodocecane, cyclomethicone, and the like. These may or may not evaporate and may or may not absorb into the skin following application.

### Amount of Active Ingredient

Many active ingredients are used at fairly low levels, generally in amounts less than the polymer that is present. There is no limit, however, other than that the active ingredients need to be present in amounts that are useful, and that both the liquid composition to be applied to the skin and the dried film be stable during their expected lifetimes. The amount of active ingredient may be determined by those of skill in the art and would depend on the potency of the specific active ingredient(s), on the specific polymer, and the compatibility of the polymer and the active ingredient, and the rate of transfer of the active ingredient into the adjacent skin from the film composition.

A guideline for a maximum amount of active ingredient that should be present in proportion to the polymer is that a dry film should be formed. If too much of an active ingredient were present the film formed may be sticky, tacky or too soft to have a desired integrity. Also, a sign of too much active ingredient would be the observation that the active ingredient was exuding from the polymer film and forming an undesirable trait such as a greasy feel, a messy, wet feeling film, an oily film, and the like. Thus, the active ingredient and polymer should have a degree of compatibility and not have substantial greasiness, oiliness, etc. Transparent or hazy films are suitable. Another limit for the maximum amount of active ingredient present in the film would be the active's interference with the adhesion of the film to the skin.

A minimum amount of active ingredient would be that which would deliver to the skin a useful amount of the active ingredient over the intended time of film contact with the skin. This is dependant on the active ingredient, the polymer composition, other formulation ingredients and their compatibility and affinity for one another versus the skin. A minimum amount of active ingredient would be the minimum effective amount, meaning that enough of the active ingredient would transfer from the film to the skin to have the desired beneficial effect.

The active ingredient incorporated into the polymer formulation may be present at form about 0.01- about 50 wt. %, from about 0.1% to about 45%, or even from about 1% to 40 wt. % of total solids of the polymer. It is desirable to employ polymer compositions having both refractive indices close to 1.55, and active ingredients that do not interfere with the refractive index, meaning that the resultant combination of ingredients when dry on the skin is close to 1.55. An effective amount of active ingredient is needed for the purpose intended.

### Suitable Active Ingredients

These may be botanical extracts: oil-soluble, glycol-soluble, or water-soluble. Examples are Aloe extract, cinnamon oil, Linden oil, avocado oil, green tea extract, Chamomile extract, sweet almond nut oil, olive oil, grape seed extract, rice bran extract, and the like. Emollients and hydrocarbon blends are suitable (for example, petrolatum, mineral oil, and the like of various molecular weights with limited volatility, such that most of the ingredient is transferred to the skin and a minimum amount is lost to evaporation). Also suitable are drugs typically transferred to the skin via patches, oils from plants, vitamins, silicones, proteins, peptides, sterols, phytosterols, amino acids, and the like. In general, the active ingredients boiling point would be about 100 degrees Celsius or greater. Preferred ingredients absorb into the skin with purpose of moisturizing, softening, nourishing, and in general promoting a sense of well-being by the applicant. Also, ingredients may be added to the composition that make the feel of the applied liquid on the skin more pleasant, such as ethanol, cyclomethicone, emollients, and the like.

Virtually any ingredient that is applied to the skin can be delivered in the composition of this invention. Thus, alpha hydroxy acids, salicylic acid, propylene glycol, glycerin, esters, petrolatum, hydroquinone, masked hydroquinone compounds and other skin lightening agents, plant abstracts, animal extracts, waxes, drugs and drug-like substances which can give some benefit via skin absorption are acceptable active ingredients.

### Suitable Polymers

Many types of polymers may be used to form films of suitable refractive index. Both synthetic and natural polymers are suitable. These polymers should also be adherent to the skin and should in general not be significantly absorbable into the skin. The suitable polymer types include uncrosslinked polymers and lightly crosslinked polymers. Polymer types include polyesters, acrylics, acrylamides, polypeptides, polyalkylene glycols, cellulose derivatives, polyurethanes, silicones, polyepoxides, polyolefins, and the like. Water soluble or water dispersible polymers, having refractive indices of from about 1.4 to 1.7, between 1.45 and 1.65, or even between 1.5 and 1.6 are suitable.

Particularly suitable polymers that meet the requirements are those known as Eastman AQ polymers. They have refractive indices of approximately 1.55, and are stabilized in aqueous dispersion by pendant sodium sulfo moieties. These and similar polymers may be used alone or in conjunction with plasticizers and surfactants to incorporate active ingredients into a film which may be removed both by peeling and washing. Blends with other polymers are acceptable and may provide advantage in boosting properties of the film or adjusting refractive index of the film. Blends of AQ polymers are suitable.

Suitable acrylic polymers are those meeting the refractive index criteria and having greater than about 30 weight % of an aromatic monomer, such as styrene, alpha-methyl styrene, vinyl naphthalene, benzyl methacrylate, benzyl acrylate, phenoxyethyl acrylate, phenoxyethyl methacrylate, phenoxypropyl acrylate, and the like. Other suitably monomers may be used that provide a high refractive index component of the polymer such as those containing halogen. Even higher percentages of aromatic or other monomers giving high refractive index polymer, for example 40% of the monomer, or even 50% of the monomer or more as needed to achieve the target refractive index. Tg's of such polymers should be below 80° C., below 70° C., or even below 60° C. Plasticizers may be incorporated into a formulation containing the acrylic polymer such that its effective Tg is lowered to at least 40 degrees C.

Also particularly suitable are acrylic latex polymers, especially those derived from a mini-emulsion process. Suitable monomers must be used to achieve the desired refractive index while maintaining the desired Tg, elongation, etc. These properties may be adjusted by additives to achieve the desired film refractive index and film properties. Whereas a single monomer may be polymerized using a free radical initiator to provide a polymer of the desired refractive index, the polymer properties may not be desirable for elongation or adhesion to skin, or may have poor plasticizer compatibility, and the like. It is much more common to adjust the polymer refractive index and the other desired polymer properties by polymerizing mixtures of monomers such as, for example, 2-ethylhexyl acrylate, styrene, and an unsaturated acid such as acrylic acid. Introducing three or more monomers provide the polymer maker with even more flexibility in achieving both the desired polymer film RI and other properties such as film elongation and adhesion to skin. One skilled in the art of acrylic emulsion polymerization and coatings can provide polymers such that all requirements are met for skin adhesion, elongation, and refractive index.

Suitable polyesters are those generally having a high proportion (greater than about 30%) of aromatic monomer moiety included. A monomer moiety is that part of the original monomer remaining after the reaction occurs to join the monomers into the polymer. Examples of polyester monomers meeting these criteria of being aromatic are terephthalic acid, dimethyl terephthalate, isophthalic acid, sodium sulfo isophthalate, dimethyl terephthalate, phthalic anhydride, phthalic acid, bis-phenol derivatives such as bis-phenol A ethoxylate, bis-phenol F propoxylate, bis hydroxymethyl benzene, bis hydroxyethyl resorcinol, and the like. Particularly suitable polymers are those commercially available and known as Eastman AQ® polymers. These are polyester polymers which may be dispersed into water using only mild agitation and/or heat. These have a refractive index similar to skin at about 1.55, are adherent to the skin and form films on the skin, and are removable by peeling or washing with water.

Particularly suitable plasticizers which may be used with these polymers are the following: triethyl citrate, triacetin, propylene carbonate, glycerin, propylene glycol, phenoxyethanol, benzyl alcohol, lactic acid, lactamide, glycolic acid, acetoxytriethyl citrate, monoglycerides, diethyl citrate, diethyl phthalate, diethyl terephthalate, diethyl isophthalate, dipropyl isophthalate, ascorbic acid and its esters, tartaric acid and its esters, and the like. These may be used alone or in combination with others. Generally those organic materials with between about 20% and 65% oxygen by weight oxygen, or the same 20-65% oxygen plus nitrogen, (further referred to hereinafter as % heteroatoms), may be suitable. Greater than 30% heteroatom content is preferred, and even more preferable is more than 40% heteroatoms. Often, generally for small molecule plasticizers of molecular weight of less than about 300, 50% to about 65% heteroatom is acceptable. Note that the composition of the polymer, its refractive index, and the respective refractive index of the plasticizer are important to consider when preparing a formula. Since the plasticizer and polymer mix intimately, there will be a combined refractive index influence on the final film if intimate mixing has occurred. The refractive index of the mixture when dry on the skin should approximately match the refractive index of the skin.

Polymers may be water soluble or water dispersible, or may be solvent soluble or solvent dispersible. Those polymers delivered in wet form (i.e. those that dry on the skin from an applied liquid) may be delivered from water, alcohol or other organic solvent not harmful to the skin, or mixtures thereof. Many such solvents are available to cosmetic chemists, from ethanol, isododecane, hydrophobic esters such as glycerol monooleate, mineral oil, and the like. The solvent may evaporate after application to the skin, or soak into the skin if the solvent has limited volatility. Polymers designed to be removed by peeling may be applied from any solvent including water. For polymers designed to be removed by washing or exposing to water may be applied from any solvent, although preferably from water or water in combination with other solvents compatible with water.

Suitable water solubilizing groups, or groups which promote water dispersion of polymers include sulfonate and sulfonate salts, sulfate and sulfate salts, carboxylic acid salts, phosphate and phosphate salts, amine salts, quaternary ammonium, phosphonium salts, and the like. Combinations may be used. Polymers may be negatively changed, positively charged, or neutral, or amphoteric.

For most applications, a degree of water resistance is desired in the film in contact with the skin which contains the active ingredient for delivery. The film should be removable by peeling or washing with water or soap and water. The film should have a degree of abrasion resistance when dry, however, it should be removable when wet with water by rubbing. Soap may assist in the film removal.

### Film Thickness

In general, a thin film is best. Film thickness from about 0.1 mils (2.5 micrometers) to about 10 mils (250 micrometers) is suitable. A film thickness of from about 0.5 to about 5 mils (12.5 to about 125 micrometers) or even from about 1 to about 4 mils (25 to 100 micrometers) is suitable. Film thickness is one of the determining factors as to how much active ingredient can be contained in a film. The film may be tapered on its edges after application and while still wet by rubbing with the finger so that the film edge will not be as noticeable. Compatibility of the active ingredient and polymer is another factor. Refractive index, as mentioned earlier, is effected not only by the inherent refractive index of the polymer but also the added ingredients that are in intimate contact with the polymer and become part of the dried film. Water retained by the polymer may also influence the film refractive index. Those ingredients not intimately mixed and existing in separate phases may not significantly impact the film refractive index, but may serve to render the film somewhat opaque or reduce the film gloss as mentioned earlier.

### Film Elasticity

The film containing an active ingredient when in contact with the skin should remain flexible, having an elongation of at least 50%, at least 100%, or even at least 150% for the duration desired on the skin. Elongation of 200% or more is acceptable as well. This property of the film enables the film to stretch to conform to and maintain adhesion to without tearing, areas of the skin such as elbows, knuckles on fingers, on the face, around the eyes, and the like. An added feature of this enhanced elongation is that the thin films when in contact with the skin are imperceptible on the skin. That is, the films cannot be perceived by feeling of the skin surface in contact with the film.

### Additives, Film Characteristics

Many other additives may be contained within the film. Solvents, plasticizers, flatting agents, particulate materials, vitamins, surfactants, gloss reducing agents, emollients, peptides, lipids, dyes, pigments, ultraviolet absorbing materials, antioxidants, chelating agents, lubricants, silicone oligomers or polymers, hydrocarbons, esters, ketones, alcohols, and the like may be added for purposes such as to change the appearance of the film (e.g. from shiny to flat), to provide a cooling effect, to provide a better feel on application, to stabilize the ingredients, to render the film more easily removable from the skin, and the like.

### EXAMPLES

### Example 1

### Preparation of Sulfopolyester A

A round bottom flask equipped with ground-glass head, an agitator shaft, nitrogen inlet and a side arm was charged with 82 mole percent isophthalic acid, 18 mole percent dimethyl-5-sodiosulfoisophthalate (SIP), 54 mole percent diethylene glycol (DEG), and 46 mole percent 1,4-cyclohexanedimethanol (CHDM), based on 100 mole percent dicarboxylic acid and 100 mole percent diol. A catalyst was added and the flask was immersed in a Belmont bath at 200° C. for one hour under a nitrogen sweep. The temperature of the bath was increased to 230° C. for one hour. After one hour the temperature of the bath was increased to 280° C. and the flask was heated for 45 minutes longer under a reduced pressure of 0.5 to 0.1 mm of Hg. The flask was allowed to cool to room temperature. The copolyester was removed from the flask and ground to less than 3 mm granules. Sulfopolyester A had a Tg of 53° C. (as determined by differential scanning calorimetery) and an Inherent Viscosity (I.V.) of 0.33 dl/g was measured at 23° C. using 0.50 grams of polymer per 100 ml of a solvent consisting of 60% by weight phenol and 40% by weight tetrachloroethane. The refractive index of the polymer was determined to be 1.5525.

A dispersion of the Sulfopolyester A polymer granules was prepared by heating to 80° C. 136 grams of deionized water in a 500 milliliter beaker. Then 64 grams of the polymer granules were added with stirring, and the stirring continued for 30 minutes. The weight of the water that evaporated on heating was replaced as the formula cooled, giving a nearly clear polymer dispersion.

### Example 2

### Preparation of Sulfopolyester B.

Following the procedure of Example 2 above Sulfopolyester B was prepared with the following exceptions: 11 mole percent dimethyl-5-sodiosulfoisophthalate and 89 mole percent isophthalic acid, and 21.5 mole percent 1,4-cyclohexanedimethanol and 78.5 mole percent diethylene glycol, based on 100 mole percent dicarboxylic acid and 100 mole percent diol. The resultant Sulfopolyester B has a Tg of 35° C. and an I.V. of 0.32 dl/g using 0.50 grams of polymer per 100 ml of a solvent consisting of 60% by weight phenol and 40% by weight tetrachloroethane. The refractive index of the polymer was 1.5547.

A dispersion of the Sulfopolyester B polymer granules was prepared by heating to 80° C., 136 grams of deionized water in a 500 milliliter beaker. Then 64 grams of the polymer granules were added with stirring, and the stirring continued for 30 minutes. The weight of the water that evaporated on heating was replaced as the formula cooled, giving a slightly turbid polymer dispersion.

### Example 3

The dispersion of Example 1 was blended with the following amounts of triethyl citrate. Then to 20 grams of each resulting dispersion, to 0.15 grams of Zonyl® FSO was added. Dry films were prepared by preparing a drawdown using a bar having an 8 mil gap on a polytetrafluoroethylene fluorocarbon substrate, and allowing the film to dry overnight. The film refractive index was then measured.

| | Dispersion of | Triethyl | Dry Film |
|---|---|---|---|
| | Example 1 | citrate | Refractive |
| Experiment | (grams) | (grams) | Index |
| A | 100 | 2.4 | 1.5475 |
| B | 100 | 4.36 | 1.5424 |
| C | 100 | 5.6 | 1.5396 |
| D | 100 | 8.0 | 1.5337 |

These experiments demonstrate the influence of the added plasticizer on the film refractive index.

### Example 4

In accordance with the present invention, a formulation was prepared by combining in a 1 ounce wide-mouth jar the following constituents: (a) 20.44 g of the dispersion from Example 2; (b) 1.2 g triacetin (available from Eastman Chemical Company); (c) 1.2 g DG Petroleum Jelly (available from Dolgen Corp., Inc., 100 Mission Ridge, Boodlettsville, Tenn. 37072); and (d) 0.47 g Clearate Lecithin emulsifier (available from W.A. Cleary Corp., 1049 Route 27, P.O. Box 10, Somerset, N.J. 08875-0100). The bottle was placed in a water-bath at 80° C. for 1 hour. The bottle was removed, and was shaken rapidly on a Brinkman Vibratory Mill until it was cool. The emulsion was creamy and did not separate upon standing.

The formulation formed a film in less than 5 minutes when an amount was brushed to the back of a test subject's hand and allowed to dry. The film was not greasy to the touch, and was not tacky to the touch after the 5 minutes drying time. After 2 hours, the film was removed from the test subject's hand by washing with water. The skin beneath the spot where the film had resided felt smooth to the touch.

The formulation was drawn down on a release film (Polyester Liner L-25X available from Sil-Tech, 222 Mound Avenue, Miamisburg, Ohio 45342) using a 4 mil (0.004 of an inch) gap film applicator which deposited an approximately 2 mil (0.002 inch) thick wet film. The coating after being allowed to dry at ambient temperature overnight had an elongation of greater than 600% as measured by ASTM Method D882. The dry film before elongation had a thickness of 0.66 mil (0.000066 of an inch) demonstrating the high flexibility of the film).

### Example 5

The preparation of Example 4 was used to demonstrate the improvement in wrinkled skin appearance. The composition was applied using a small, flexible-bristle brush to the skin surrounding the eye of a female volunteer of the approximate age of 50 and allowed to dry. Observations were made before application, at 5 minutes and every five minutes thereafter for 15 minutes following the application. The film was then removed by washing with tap water (no soap), then the skin was dried, and final observations of the skin were made.

Dramatic lessening of wrinkles was observed as the film dried. Even though the film was shiny when dry on the skin, it was obvious to the observer that fewer and shallower wrinkles were seen after the composition application and film drying than was present before film application. Many of the smaller wrinkles appeared to completely disappear. Upon film removal, observations indicated that wrinkles were less intense than prior to application. Similar testing was conducted on two different female volunteers with the same results and conclusions.

### Example 6

A formulation was prepared by blending: a) 335.24 grams of a 32% aqueous dispersion of the polymer of Example 2; b) 20.74 grams triacetin; c) 10.56 grams lecithin; d) 26.91 grams petrolatum; and e) 4.17 grams of a 2% aqueous solution of EDTA.2Na.H2O. When all the solids were added, high shear mixing was continued until a stable dispersion was produced. To 75.25 grams of this blend was added 4.01 grams of ACEMATT® OK412 precipitated silica, (available from Degussa) which was stirred in by hand using a wooden tongue depressor. When the viscous blend was applied to the skin on the wrist or knuckles, the dried film was not visually apparent. The system appeared to fill wrinkles so that their appearance was diminished. It was also observed that resistance of the dry film to crack on the skin was improved relative to an identical film without the silica. A drawdown using a bar with a 3 mil gap of the same mixture on an aluminum Q-panel gave a 60° gloss measurement of 10.2, versus the gloss of the control film of 62.6, indicating that a substantial reduction in film gloss has occurred by adding the silica.

### Example 7

To a 1000 ml resin kettle equipped with a condenser, nitrogen purge, and a subsurface feed tube was added 400 g of water, 1.6 g of sodium dodecyl sulfate surfactant and 5.7 g of Eumulgin B2PH surfactant (available from Cognis). A nitrogen purge was begun and the mixture was stirred at 200 rpm while heating the contents to 80° C.

In a separate flask were mixed 0.67 g of sodium dodecyl sulfate, 1.4 g of Eumulgin B2PH surfactants and 185 g of water. Monomer pre-emulsion was prepared by adding to this water surfactant mixture, 296 g of monomers consisting of styrene/2-ethylhexyl acrylate/benzyl acrylate/methacrylic acid in ratio of 35.9/15.3/39.0/9.8 respectively. The mixture was stirred at room temperature for 30 minutes to obtain a stable milky looking pre-emulsion. 0.3 g of 3-mercapto-1,2 propane diol was also added to this mixture as chain transfer agent.

Thirty-four (34) grams of pre-emulsion was charged to the reactor. Then 0.2 g of ammonium persulfate was mixed in 10 g of water and charged to the reactor mixture, still held at 80° C. After 15 minutes, the remaining pre-emulsion was fed over a period of 120 minutes to the reactor. Simultaneously, an initiator feed composed of 75.0 g of water and 0.32 g of ammonium persulfate was also fed to the reactor over the time period of 135 minutes. After the feeds ended, the reactor was held at 80° C. for additional 30 minutes. Then a reductant solution consisting of 5.0 g water and 0.06 g of ascorbic acid was added to the reactor. A solution of 20.0 g water and 0.2 g of 30% hydrogen peroxide was then fed to the reactor over 120 minutes. The reaction mix was cooled to room temperature. The latex was filtered through a 100 mesh wire screen and filterable solids or scrap was determined as less than 0.1% based on the total batch weight. The particle size was measured using Microtrac UPA Particle Size Analyzer—laser light-scattering device (180 degree backscattering). For this particle size measurement the sample was diluted approximately 1:50 in water. Resulting latex had the following properties. Solids: 31.475, Viscosity (Sp2@60 rpm): 7 cps, pH 3.58, Average Particle size: 65 nm, Refractive index of a film dried in an oven at 50 degrees C., 1.558 (calculated 1.55), Tg: 42 C (calculated 33 C), residual monomers: 18 ppm.

### Example 8

### Mini-Emulsion (Tg+5) with 10% Petrolatum and COFA

To a 1000 mL resin kettle equipped with a condenser, nitrogen purge, and a subsurface feed tube was added 120 g of water. A nitrogen purge was begun and the contents heated and maintained at 80° C. Coconut Oil Fatty Acid (COFA), 41 grams, (C-108 obtained from Proctor and Gamble) was preheated at 60° C. and mixed with 41 grams of pre-heated (60° C.) petrolatum (purchased as Petroleum Jelly). The COFA-Petrolatum mixture in this example contained 10% Petrolatum (based on the weight of the total monomers).

This viscous liquid mixture was slowly added under stirring to a monomer mix consisting of 415.0 grams styrene/2-ethylhexyl acrylate/acetoacetoxy ethylmethacrylate/methacylic acid/acrylic acid. The weight ratio of monomers in the monomer mix was 44.5/43.2/9.4/0.7/2.2, respectively.

Water (365 grams) and 18.3 grams of a surfactant mixture (Aerosol OT-NV (available from Cytec Industries) and Hitenol BC1025 (available from DKS) in ratio of 1.1:0.4.) were premixed. The monomer/Petrolatum/COFA mixture was then added to form a pre-emulsion. The pre-emulsion was sheared using an IKA (Model SD-45) rotor/stator homogenizer by pumping through a flow cell which surrounded the shearing device with the homogenizer operating at maximum rpm to form a miniemulsion. Seventy six (76) grams of the miniemulsion was charged to a reactor. Then 0.6 g of ammonium persulfate was mixed in 10 g of water and charged to the reactor mixture, still held at 80° C. After 15 minutes, the remaining miniemulsion was fed over a period of 180 minutes to the reactor.

Simultaneously, an initiator feed composed of 79.0 g of water, 0.84 g of ammonium persulfate, and 0.84 g of ammonium carbonate was also fed to the reactor over the time period of 180 minutes. After the feeds ended, the reactor was held at 80° C. for 60 minutes. Afterwards the reactor mixture was cooled to 50° C. Then a reductant solution consisting of 6.4 g water, 1.0 g isoascorbic acid, and 1.2 g of 0.5% iron sulfate heptahydrate, and 0.34 g of 28% ammonium hydroxide was added to the reactor. A solution of 19.0 g water and 1.10 g 70% t-butyl hydroperoxide was then fed to the reactor over 48 minutes. The reaction mix was cooled to room temperature. The latex was filtered through a 100 mesh wire screen and filterable solids or scrap was determined as less than 0.1% based on the total batch weight. The droplet and particle sizes were measured using Microtrac UPA Particle Size Analyzer—laser light-scattering device (180 degree backscattering). For this particle size measurement the sample was diluted approximately 1:50 in water.

A free film was prepared using a 5 mil gap drawbar on a fluorocarbon release substrate, letting the film dry at ambient temperature overnight, then separating the film from the substrate. The refractive index of the dry film was 1.515, vs that predicted by linearly combining the refractive indices of the ingredients of 1.512.

### Example 9

### Mini-Emulsion (Tg+5) with 20% Petrolatum and 10% COFA

To a 1000 mL resin kettle equipped with a condenser, nitrogen purge, and a subsurface feed tube was added 120 g of water. A nitrogen purge was begun and the contents heated and maintained at 80° C. Coconut Oil Fatty Acid (COFA), 41 grams, (C-108 obtained from Proctor and Gamble) was preheated at 60° C. and mixed with 82 grams of pre-heated (60° C.) petrolatum (purchased as Petroleum Jelly). The COFA-Petrolatum mixture in this example contained 20% Petrolatum (based on the weight of the total monomers).

This viscous liquid mixture was slowly added under stirring to a monomer mix consisting of 415.0 grams styrene/2-ethylhexyl acrylate/acetoacetoxy ethylmethacrylate/methacylic acid/acrylic acid. The weight ratio of monomers in the monomer mix was 44.5/43.2/9.4/0.7/2.2, respectively.

Water (365 grams)and 18.3 grams of a surfactant mixture (Aerosol OT-NV (available from Cytec Industries) and Hitenol BC1025 (available from DKS) in ratio of 1.1:0.4.) were premixed. The monomer/Petrolatum/COFA mixture was then added to form a pre-emulsion. The pre-emulsion was sheared using an IKA (Model SD-45) rotor/stator homogenizer by pumping through a flow cell which surrounded the shearing device with the homogenizer operating at maximum rpm to form a miniemulsion. Seventy two (72) grams of the miniemulsion was charged to a reactor. Then 0.6 g of ammonium persulfate was mixed in 10 g of water and charged to the reactor mixture, still held at 80° C. After 15 minutes, the remaining miniemulsion was fed over a period of 180 minutes to the reactor. Simultaneously, an initiator feed composed of 78.0 g of water, 0.85 g of ammonium persulfate, and 0.85 g of ammonium carbonate was also fed to the reactor over the time period of 180 minutes. After the feeds ended, the reactor was held at 80° C. for 60 minutes. Afterwards the reactor mixture was cooled to 50□C. Then a reductant solution consisting of 7.0 g water, 1.0 g isoascorbic acid, and 1.2 g of 0.5% iron sulfate heptahydrate, and 0.34 g of 28% ammonium hydroxide was added to the reactor. A solution of 20.0 g water and 1.10 g 70% t-butyl hydroperoxide was then fed to the reactor over 48 minutes. The reaction mix was cooled to room temperature.

The latex was filtered through a 100 mesh wire screen and filterable solids or scrap was determined as less than 0.1% based on the total batch weight. The droplet and particle sizes were measured using Microtrac UPA Particle Size Analyzer—laser light-scattering device (180 degree backscattering). For this particle size measurement the sample was diluted approximately 1:50 in water. A film was prepared by first blending 40.0 grams of the emulsion with 1.05 grams of Purethix HH (available from Sud Chemie) making a drawdown using a drawbar with a gap of 5 mils. The film was allowed to dry overnight under ambient conditions, then separated from the fluorocarbon substrate on which it was prepared. The refractive index of the dry film was determined as 1.513, vs that predicted by linearly combining the refractive indices of the ingredients of 1.506.

Since the calculated refractive index is close to that predicted for Examples 7 and 8, and may vary slightly due to unaccounted variables such as the polymeric thickener content (for which the refractive index is unknown and therefore ignored in the calculation), the linear combination of refractive index components appears very predictive.

### Example 10

### Emulsion Polymer X28645-162

To a 1000 ml resin kettle equipped with a condenser, nitrogen purge, and a subsurface feed tube was added 400 g of water, 1.6 g of sodium dodecyl sulfate surfactant and 5.6 g of Eumulgin B2PH surfactant. A nitrogen purge was begun and the mixture was stirred at 200 rpm while heating the contents to 82° C.

In a separate flask were mixed 0.67 g of sodium dodecyl sulfate, 1.4 g of Eumulgin B2PH surfactants and 185 g of water. A monomer pre-emulsion was prepared by adding to this water surfactant mixture, 274 g of monomers consisting of ethyl acrylate/methacrylic acid in ratio of 52.6/47.4, respectively. The mixture was stirred at room temperature for 30 minutes to obtain a stable milky looking pre-emulsion. 0.6 g of 3-mercapto-1,2 propanediol was also added to this mixture as chain transfer agent.

A monomer mix consisting of 4.3 g of ethyl acrylate and 4.2 g of methacrylic acid was prepared in a small beaker and was charged to the reactor when the reactor temperature was 80° C. Then 0.2 g of ammonium persulfate was mixed in 10 g of water and charged to the reactor mixture, still held at 80° C. After 15 minutes, pre-emulsion prepared above was fed over a period of 120 minutes to the reactor at 82° C. Simultaneously, an initiator feed composed of 75.0 g of water and 0.32 g of ammonium persulfate was also fed to the reactor over the time period of 165 minutes. At end of pre-emulsion feed after 120 minutes, both feed pumps were stopped. The pre-emulsion reservoir was charged with additional 13.1 g of methacrylic acid and was fed to the reactor over 35 minutes and initiator feed was also resumed at this time. After the feeds ended, the reactor was held at 82° C. for additional 30 minutes. Then a reductant solution consisting of 5.0 g water and 0.06 g of ascorbic acid was added to the reactor. A solution of 20.0 g water and 0.2 g of 30% hydrogen peroxide was then fed to the reactor over 120 minutes. Reaction mixture was held at 82° C. for additional 2 hours. The reaction mix was cooled to room temperature. The latex was filtered through a 100 mesh wire screen and filterable solids or scrap was determined as less than 0.1% based on the total batch weight. The particle size was measured using Microtrac UPA Particle Size Analyzer—laser light-scattering device (180 degree backscattering). For this particle size measurement the sample was diluted approximately 1:50 in water. Resulting latex had the following properties. Solids: 30.7%, Viscosity (Sp1@60 rpm): 9 cps, pH 3.58, Average Particle size: 107 nm, Refractive index; calculated 1.49, Tg: calculated 55 C, residual monomers: 7 ppm.

### Example 11

### Comparison of Dispersion from Examples 7 and 10

Each polymer dispersion from Example 7 (film RI=1.558) and from Example 10 (film RI=1.49) were individually prepared for film formation by adding triethyl citrate plasticizer. To 10 grams of each polymer dispersion (after the emulsion had been aged so that any residual monomer was below acceptable levels) was added 0.45 g of triethyl citrate. Each blended sample in a vial was shaken vigorously to mix, then placed in a 50 degree C. oven for 1.25 hours, then removed from the oven to cool the sample.

After cooling 1.25 hours, each plasticized latex sample was applied to the knuckle of a male volunteer with a small flexible brush—each on the same knuckle, but on different sides, so that the main lines of the wrinkle ran through coated areas of each latex film. Then the next knuckle was treated similarly, except that each liquid was applied on the opposite side of the knuckle to that done in the previous application. The films dried rapidly, well within 5 minutes. Although the film from the Example 7 latex was glossier than that from Example 10, both had gloss significantly higher than the surrounding untreated skin. While both polymer films lessened the appearance of the wrinkle depth and breadth relative to the surrounding untreated skin, the film formed from the Example 7 latex minimized the appearance even more, especially of the smaller wrinkles.

The invention has been described in detail with particular reference to preferred embodiments thereof, but it will be understood that variations and modifications can be effected within the spirit and scope of the invention.

We claim:

1. A liquid cosmetic composition, comprising:
at least 5% by weight of a film forming polymer; and
a cosmetically acceptable carrier, wherein, after the composition is applied to and dried on skin, the composition has a refractive index of between about 1.4 and about 1.7.

2. The composition according to claim 1, wherein the refractive index is between about 1.5 and about 1.6.

3. The composition according to claim 2, wherein the refractive index is about 1.55.

4. The composition according to claim 1, wherein said polymer is at least one of a sulfopolyester, a polyesteramide, an acrylic polymer, and a polyurethane.

5. The composition of claim 4, wherein the acrylic polymer is prepared from at least one of benzyl acrylate, phenoxyethyl acrylate, benzyl methacrylate, and phenoxyethyl methacrylate.

6. The composition according to claim 1, wherein said composition includes between about 5% to about 50% by weight of the polymer.

7. The composition according to claim 6, wherein said composition includes between about 10% to about 40% by weight of the polymer.

8. The composition according to claim 7, wherein said composition includes between about 15% to about 35% by weight of the polymer.

9. The composition according to claim 1, wherein said composition further comprises at least one of particulate material, pigment and dye.

10. The composition to claim 9, wherein the particulate material is from about 0.1 to about 250 micrometers in diameter.

11. The composition according to claim 1, wherein said composition further comprises at least one of an active ingredient, solvent and plasticizer.

12. The composition according to claim 1, wherein said composition further comprises at least one ingredient selected from the group consisting of active ingredients, plasticizers, coalescing agents, solvents, oils, emollients, gloss reducing agents, humectants, fillers and fragrances.

13. The composition according to claim 1, wherein said composition further comprises a surfactant

14. The composition according to claim 1, wherein said composition is an emulsion.

15. The composition according to claim 1, wherein said cosmetically acceptable carrier is selected from the group consisting of water, ethanol, isododecane, cyclomethicone, and mixtures thereof.

16. The composition according to claim 1, wherein the composition is a gel or paste.

17. A method of making a cosmetic composition, comprising:
combining a film forming polymer and a cosmetically acceptable carrier to form the cosmetic composition,
wherein the film forming polymer is present in an amount of at least 5% by weight, and
after the composition is applied to skin and dries, the composition has an RI of between about 1.4 and about 1.7.

18. The method according to claim 17, wherein an active ingredient is also combined with the polymer and carrier.

19. The method according to claim 17, wherein at least one ingredient gloss, reducing agents, oils, emollients, humectants, pigments, fillers, and fragrance is also combined with the polymer and carrier.

20. The method according to claim 17, wherein one of an emulsifier and surfactant is combined with the polymer and carrier.

21. The method according to claim 17, wherein composition is an emulsion.

22. A method of using a cosmetic composition, comprising applying the composition of claim 1 to skin to thereby form a film on said skin.

23. A method according to claim 22, further comprising tapering edges of said film.

24. A method according to claim 22, wherein said film has a thickness of between about 2.5 μm to about 250 μm.

25. A method according to claim 24, wherein said film has a thickness of between about 12.5 μm to about 125 μm.

26. A method according to claim 25, wherein said film has a thickness of between about 25 μm to about 100 μm.

27. A method according to claim 22, wherein said film contains particulates of diameter from about 1% to about 100% of the thickness of the dried film.
